(19) United States
(12) Patent Application Publication
(10) Pub. No.: US 2026/0301915 A1
(43) Pub. Date: Oct. 1, 2026

(54) RESPIRATORY THERAPY USAGE PREDICTIONS USING CONVOLUTIONAL NEURAL NETWORKS

(71) Applicant: ResMed Digital Health Inc., San Diego, CA (US)

(72) Inventors: Jiaming CHEN, San Diego, CA (US); Arash MEHRABAN, Anaheim, CA (US); Tom VAN DAAL, San Marcos, CA (US); Pratik Jagdishbhai AGRAWAL, Stevenson Ranch, CA (US)

(21) Appl. No.: 19/629,698

(22) Filed: Mar. 26, 2026

Related U.S. Application Data
(60) Provisional application No. 63/779,501, filed on Mar. 28, 2025.

(51) Int. Cl. G16H 20/40; G16H 10/60

(52) U.S. Cl. CPC G16H 20/40; G16H 10/60

(57) ABSTRACT

Techniques for improved machine learning are provided. A set of usage data of a user is accessed, the set of usage data comprising one or more attributes of use of a therapy device, by the user, over a plurality of prior days. An input tensor is generated based on the set of usage data, where each channel in the input tensor corresponds to a respective attribute of the one or more attributes, a first spatial dimension of the input tensor corresponds to the plurality of prior days, and a second spatial dimension of the input tensor corresponds to a plurality of windows of time within the plurality of prior days. A usage prediction is generated based on processing the input tensor using a convolutional neural network, and intervention is facilitated for the user based on the usage prediction.

### CROSS-REFERENCE TO RELATED APPLICATIONS

This application claims priority to U.S. Provisional Patent Application No. 63/779,501, filed Mar. 28, 2025, the entire content of which is incorporated herein by reference in its entirety.

### TECHNICAL FIELD

The present disclosure relates generally to machine learning, and more particularly, to use of machine learning to predict usage of respiratory therapy devices.

Many individuals suffer from sleep-related and/or respiratory-related disorders such as, for example, Periodic Limb Movement Disorder (PLMD), Restless Leg Syndrome (RLS), Sleep-Disordered Breathing (SDB) such as Obstructive Sleep Apnea (OSA) and Central Sleep Apnea (CSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These disorders are often treated using respiratory therapy systems.

Each respiratory therapy system generally has a respiratory therapy device connected to a user interface (e.g., a mask) via a conduit and optionally a connector. The user wears the user interface and is supplied a flow of pressurized air from the respiratory therapy device via the conduit. In some cases, patient usage of the therapy system can be collected or monitored in order to evaluate user compliance with the therapy. For example, the duration of usage (e.g., the length of time that a user wears the user interface on a given night) may be determined. Generally, a wide variety of factors may affect compliance and usage. Although many patients would benefit from increased therapy usage (e.g., using their mask for a longer period of time each night), it is generally difficult or impossible to effectively predict and improve usage.

Improved systems and techniques to predict usage and thereby improve therapy and outcomes are desired.

### SUMMARY

According to some implementations of the present disclosure, a method includes: accessing a set of usage data of a user, the set of usage data comprising one or more attributes of use of a therapy device, by the user, over a plurality of prior days; generating an input tensor based on the set of usage data, wherein: each channel in the input tensor corresponds to a respective attribute of the one or more attributes, a first spatial dimension of the input tensor corresponds to the plurality of prior days, and a second spatial dimension of the input tensor corresponds to a plurality of windows of time within the plurality of prior days; generating a usage prediction based on processing the input tensor using a convolutional neural network; and facilitating intervention for the user based on the usage prediction.

According to some implementations of the present disclosure, a system includes a control system and a memory. The control system includes one or more processors. The memory has stored thereon machine readable instructions. The control system is coupled to the memory, and any one of the methods disclosed herein is implemented when the machine executable instructions in the memory are executed by at least one of the one or more processors of the control system.

Other aspects provide processing systems configured to perform the aforementioned method as well as those described herein; non-transitory, computer-readable media comprising instructions that, when executed by one or more processors of a processing system, cause the processing system to perform the aforementioned methods as well as those described herein; a computer program product embodied on a computer-readable storage medium comprising code for performing the aforementioned methods as well as those further described herein; and a processing system comprising means for performing the aforementioned methods as well as those further described herein.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 depicts an example workflow for predicting therapy usage and facilitating appropriate intervention, according to some embodiments of the present disclosure.

FIG. 2 depicts an example workflow for generating input tensors based on granular usage data, according to some embodiments of the present disclosure.

FIG. 3 depicts example timelines of usage predictions, according to some embodiments of the present disclosure.

FIG. 4 is a flow diagram depicting an example method for training machine learning models to predict therapy usage, according to some embodiments of the present disclosure.

FIG. 5 is a flow diagram depicting an example method for using machine learning models to predict therapy usage, according to some embodiments of the present disclosure.

FIG. 6 is a flow diagram depicting an example method for generating granular input tensors, according to some embodiments of the present disclosure.

FIG. 7 is a flow diagram depicting an example method for predicting usage using machine learning, according to some embodiments of the present disclosure.

FIG. 8 depicts an example computing device configured to perform various aspects of the present disclosure, according to some embodiments disclosed herein.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Embodiments of the present disclosure generally provide techniques for using machine learning to predict therapy device usage.

Generally, respiratory therapy refers to the use of a flow generator and user interface to deliver air and/or oxygen to a user, such as during sleep. For example, respiratory therapy may include use of continuous positive airway pressure (CPAP) devices, bi-level positive airway pressure (BiPAP) devices, and the like. Respiratory therapy can significantly improve the lives of users who engage in it. However, many users do not use such respiratory therapy devices sufficiently (e.g., for sufficient durations and/or sufficiently often) to achieve optimal results. A wide variety of approaches may be used to improve therapy usage (e.g., to increase the length of time the user uses the flow generator each night). However, due to inherently limited resources, it may be preferable to target such efforts to users who are otherwise unlikely to engage sufficiently in the therapy (e.g., users who only use their therapy device a relatively small amount of time, or may stop entirely).

Aspects of the present disclosure provide techniques and architectures to predict therapy usage. In some embodiments, a wide variety of usage data may be collected and evaluated to generate such predictions. For example, the usage data may include features related to the timing and/or duration of each usage session (e.g., timestamps for the mask-on event, when the user donned the user interface of the respiratory therapy system, and the mask-off event, when the user doffed the interface), the patient's apnea-hypopnea index (AHI) (e.g., the number of breathing interruptions per hour during the usage session), the rate(s) of air leak (e.g., mouth leak, mask leak, and the like) during the session, the patient's tidal volume (the volume of air that moves in and/or out of the user's mouth with each breath), the patient's minute ventilation (e.g., the volume of air that moves in and/or out of the patient's lungs per minute), the respiratory rate and/or flow rate of the therapy, the heart rate of the patient, the oxygen saturation of the user, and the like.

In some aspects, the raw usage data may be highly granular (e.g., including minute-by-minute or second-by-second values for one or more of the features). Some attempts to predict usage have relied on evaluating summarized usage data (e.g., by evaluating the total duration of usage over one or more days). However, this summarization sacrifices granularity, potentially resulting in less accurate predictions. Additionally, efforts to model the usage data as a time series problem (e.g., generating the prediction based on a sequence of values for each feature) may result in relatively poor prediction accuracy for longer time frames (e.g., longer input sequences and/or predictions for times that are further in the future).

In some embodiments of the present disclosure, the usage data can be preprocessed to generate a multidimensional tensor at any desired level of granularity. As discussed in more detail below, this customizable granularity can significantly improve model accuracy while balancing computational expense of the model. Further, representing the usage data as an input tensor enables use of powerful machine learning architectures, such as convolutional neural networks (CNNs). This can enable significantly improved prediction accuracy, especially for long-term predictions.

In some embodiments of the present disclosure, some or all of the operations may be performed using one or more remote services or systems (e.g., hosted in a cloud deployment). In some embodiments, such services may be accessible via transmissions over one or more communications links or networks (e.g., the Internet). For example, in some embodiments, usage data may be provided to a remote system (e.g., using one or more application programing interfaces (APIs)) with a request that the system generate an appropriate input tensor (based on the usage data) that can be used as input to machine learning model(s) (locally or via remote resources), as discussed in more detail below. As another example, the input tensor (which may be generated locally or using a remote system) may be provided (e.g., via API call) to a system that hosts machine learning model(s), requesting that the remote system (e.g., the cloud system) generate a corresponding prediction (e.g., predicting the probability of user dropout), as discussed in more detail below.

### Example Workflow for Predicting Therapy Usage and Facilitating Appropriate Intervention

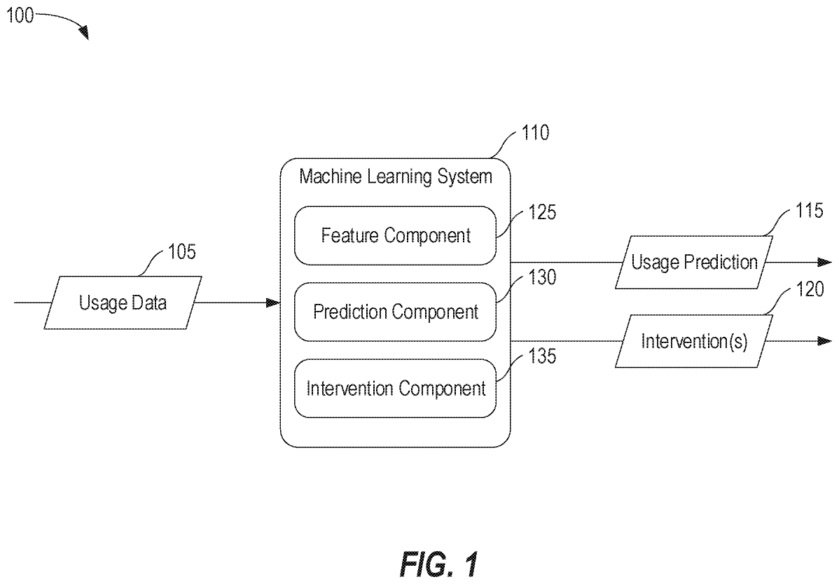

FIG. 1 depicts an example workflow 100 for predicting therapy usage and facilitating appropriate intervention, according to some embodiments of the present disclosure.

In the illustrated example, usage data 105 is accessed by a machine learning system 110 to generate usage prediction(s) 115 and/or intervention(s) 120. As used herein, “accessing” data may generally include receiving, requesting, retrieving, collecting, generating, obtaining, measuring, or otherwise gaining access to the data. For example, the machine learning system 110 may receive the usage data 105 from one or more usage data repositories, directly from the therapy system of a user, and the like.

Although a discrete machine learning system 110 is depicted for conceptual clarity, the operations of the machine learning system 110 may be combined or distributed across any number and variety of systems, and may be implemented using hardware, software, or a combination of hardware and software.

As discussed above, the usage data 105 may generally include values for one or more features related to usage of a respiratory therapy system by a user, such as when the user began and ended one or more sessions, therapy-related details of each session (e.g., AHI), and the like. In some aspects, as discussed above, some or all of the usage data 105 may have high granularity (e.g., minute-level). That is, the usage data 105 may generally include, for each of one or more features, a sequence of values (e.g., the user's tidal volume and/or minute ventilation for each minute). In some aspects, the usage data 105 includes information relating to one or more usage sessions across one or more days. For example, the usage data 105 may include the usage information for a given user for each day of the last month (e.g., the last thirty days).

In the illustrated example, the machine learning system 110 includes a feature component 125, a prediction component 130, and an intervention component 135. Although depicted as discrete components for conceptual clarity, the operations of the depicted components (and others not illustrated) may be performed by any number and variety of components, and may be implemented using hardware, software, or a combination of hardware and software.

In some embodiments, the feature component 125 may be used to preprocess the usage data 105 in order to generate a usage tensor (also referred to in some aspects as an “input tensor” and/or a “feature tensor”). In some aspects, as discussed in more detail below, the feature component 125 can generate an input tensor having a spatial dimensionality (e.g., height and width) corresponding to the number of days in the usage data 105 (or the number of days that are used as input to the model) and the number of segments (e.g., windows of time) that are used to delineate the data in each day. For example, if the usage data 105 includes data for N days (e.g., thirty days) and the machine learning system 110 delineates the data into M windows per day (e.g., twenty-four windows if hour-by-hour granularity is used, one thousand four hundred forty windows if minute-by-minute granularity is used, and the like), the feature component 125 may generate a feature tensor that is N×M in size. For example, one spatial dimension (e.g., the height) may correspond to the set of days reflected in the usage data 105 (e.g., where each row corresponds to and includes data for a corresponding day) while the other spatial dimension (e.g., the width) may correspond to the windows of time within each day (e.g., where the value in a given column is the data for a given window of time).

In some embodiments, the dimensionality of the usage tensor may depend at least in part on the number of features or attributes used by the machine learning system 110. For example, if a single feature is used (e.g., usage duration), the feature component 125 may generate a two-dimensional feature tensor where the value of each element indicates to the user's usage of the respiratory therapy system during the corresponding window of time on the corresponding day. In some embodiments, if multiple attributes are used, each channel of the tensor may correspond to a respective attribute. For example, the first channel may correspond to the usage duration, the second channel may correspond to the user's residual AHI, and the like. In some embodiments, some or all of the usage data 105 may be normalized within each bucket, as discussed in more detail below.

In the illustrated embodiment, the prediction component 130 may generally be used to process the input tensors (generated by the feature component 125) using one or more machine learning models (e.g., convolutional neural networks) to generate output usage predictions 115. In some embodiments, the prediction component 130 may use one or more models to generate one or more usage predictions 115 for one or more specified future periods and/or moments in time, as discussed in more detail below. In some embodiments, the prediction component 130 may generate a new set of usage prediction(s) 115 for a given user periodically (e.g., each morning when the user doffs their mask), whenever updated usage data 105 is available, and the like.

In some embodiments, the usage prediction 115 may include a prediction as to whether the user's usage of their respiratory therapy device will satisfy one or more defined criteria with respect to one or more future days (e.g., during one or more future windows of time). For example, the usage prediction 115 may indicate the probability that the user will be compliant (e.g., using the therapy device at least a threshold amount per day, such as least four hours per day) for the next thirty days, in two months, in six months, and the like. In some aspects, in addition to or instead of predicting future compliance or adherence, the usage prediction 115 may indicate the likelihood of total discontinuation (e.g., zero use), the predicted mean usage (e.g., the average minutes and/or hours per day, or percentage of days with at least a threshold amount of usage), and the like.

Generally, the usage predictions 115 may be used for a wide variety of operations. In the illustrated example, the usage predictions 115 may be output from the machine learning system 110. For example, the usage predictions 115 may be output to a user (e.g., a healthcare provider such as a respiratory therapist working with the patient). In some embodiments, the usage predictions 115 may be provided to one or more downstream systems for further evaluation and/or action.

In the illustrated example, the intervention component 135 may process one or more of the usage predictions 115 to optionally generate intervention(s) 120 for the user. For example, in some embodiments, the intervention component 135 may determine whether the usage prediction(s) 115 satisfy one or more criteria (e.g., whether the user is predicted to adhere to their therapy), and may generate or facilitate interventions 120 based on the prediction. As one example, if a user is predicted to be non-compliant during some future window, the intervention component 135 may facilitate interventions such as generating an alert for the user's healthcare provider, generating suggestion(s) or instruction(s) for the user to improve the probability of compliance, and the like. In some embodiments, the interventions 120 may include targeted content or assistance based on the usage predictions 115, such as facilitating the selection and/or delivery of particular resources (e.g., multimedia related to therapy, equipment or consumables used during therapy, and the like) to the user at particular times (e.g., delivering content when the user's usage is predicted to decline, delivering consumables such as replacement filters when the user is predicted to reach the lifespan of the filters based on their predicted usage, and the like).

In these ways, the machine learning system 110 can generate multidimensional tensors based on sequential usage data 105 to enable use of machine learning model architectures (e.g., CNNs) that are uniquely adapted to such data. This can result in significant improvements to model accuracy, as well as improved utilization of the input data (e.g., utilization of all, or at least more, of the usage data 105, as compared to conventional approaches). Further, by using convolution-based models, the machine learning system 110 can process the entire usage tensor at once (rather than sequentially processing records in a time-series model), which can substantially reduce the inferencing latency (e.g., the time required to generate output predictions), as well as enabling a reduced memory footprint (e.g., less memory consumed during inference) and reduced computational expense (e.g., reduced processor usage).

### Example Workflow for Generating Input Tensors Based on Granular Usage Data

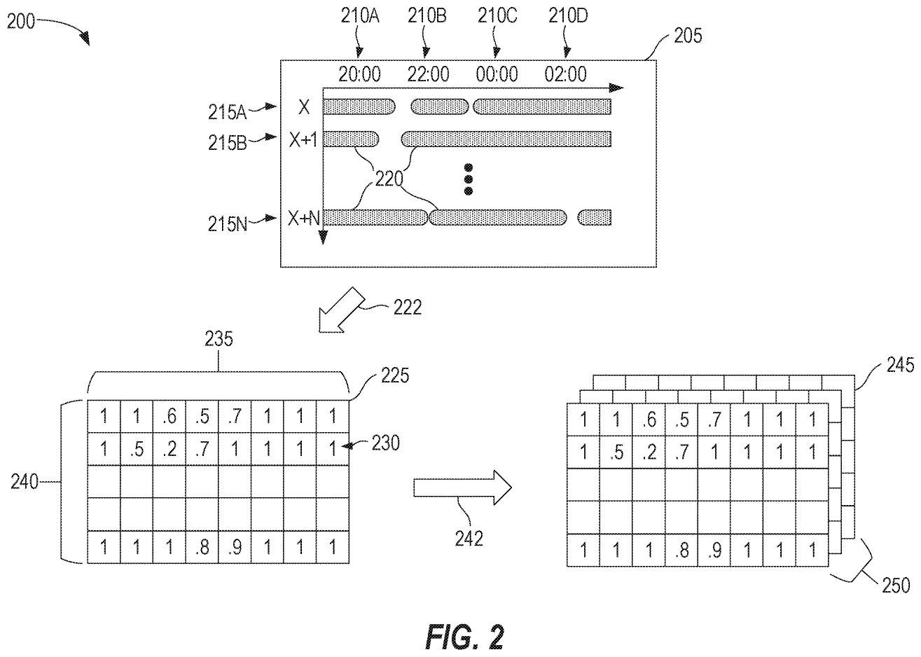

FIG. 2 depicts an example workflow 200 for generating input tensors based on granular usage data, according to some embodiments of the present disclosure. In some embodiments, the workflow 200 may be performed by a machine learning system, such as the machine learning system 110 of FIG. 1. For example, in some embodiments, the workflow 200 is performed by a feature component, such as the feature component 125 of FIG. 1.

In the illustrated workflow 200, usage data (e.g., the usage data 105 of FIG. 1) is processed to generate an input tensor 245 (also referred to as a usage tensor in some embodiments). Specifically, in the illustrated example, the usage data is depicted using a chart 205, where the usage information is depicted using shaded boxes 220 placed on the chart 205 based on the time and day corresponding to the information. Specifically, the chart 205 includes a number of rows 215A-N, where each row corresponds to a prior day (e.g., a day prior to the current day). For example, the top row 215A corresponds to day “X” (e.g., the first day in the window), the row 215B corresponds to day “X+1” (e.g., the second day), and the row 215N corresponds to day “X+N” (e.g., the Nth day in the window). As discussed above, the usage data may include information for any number of prior days (e.g., for any value of N).

Further, in the illustrated example, the horizontal axis of the chart 205 corresponds to timestamps during each day. Specifically, as illustrated, the timestamp 210A corresponds to a time of 20:00 (e.g., 8:00 PM), the timestamp 210B corresponds to 22:00 (e.g., 10:00 PM), the timestamp 210C corresponds to 00:00 (e.g., midnight), and the timestamp 210D corresponds to 02:00 (e.g., 2:00 AM). Although four timestamps 210 are illustrated for conceptual clarity, the chart 205 may generally include timestamps covering any range of time. In some embodiments, the chart 205 includes timestamps across the entire day (e.g., a twenty-four hour period for each row 215).

In the illustrated example, the chart 205 depicts usage times of the respiratory therapy system. That is, times when the user was wearing and/or using the therapy system are indicated by the boxes 220, while times the user was not wearing and/or using the therapy system are blank. For example, on day X (indicated in the row 215A), the user used the therapy device until about 21:15, at which point the user doffed the mask. The user then used the therapy system again from about 21:45 until about 23:45, then briefly stopped usage again until they restarted at about 23:50. Similarly, on day X+1 (indicated by the row 215B), the user stopped therapy at about 20:30, and restarted at about 21:30.

Although the illustrated example depicts the usage data using boxes 220 to indicate usage visually for conceptual clarity, in some embodiments, as discussed above, the usage data may include non-visual data (e.g., timestamps). That is, rather than a visual chart, the usage data may include timestamps for therapy start and therapy stop events. The feature component (or another component) may use these start and stop timestamps to determine the usage windows. For example, if the usage data includes a start event at 21:00 and a stop event at 23:00, the feature component may infer that the user used the therapy system for each moment between 21:00 and 23:00.

Generally, the start and stop events may be defined using any suitable criteria. For example, in some embodiments, the start and stop events may be defined based on when the user activated and deactivated the airflow, respectively. In some embodiments, the start and stop events may be defined based on when the user donned and doffed the mask (regardless of airflow settings), such as detected via the pressure measurements of the device. In some embodiments, the system may ignore interruptions that are less than a defined duration. For example, if the user stops therapy and then restarts less than a minute later, the system may discard this stop and start event, instead determining that the user effectively continued the therapy uninterrupted.

Although the illustrated example depicts usage times for the therapy system, as discussed above, the usage data may generally include a wide variety of information for any number of features or attributes. For example, in some embodiments, the usage data may indicate the user's AHI throughout the day across one or more days, the user's respiratory rate throughout the day, the user's tidal volume throughout the day, and the like.

In the illustrated example, as depicted by the arrow 222, the feature component can use the usage data (depicted using the chart 205) to generate a tensor 225. In the illustrated example, the rows 240 of the tensor 225 correspond to the days in the usage data (e.g., each row 240 corresponds to one of the rows 215 in the chart 205), and the columns 235 of the tensor 225 correspond to buckets or windows of time within the days (e.g., delineated based on the timestamps 210 of the chart 205). The value of each element in the tensor 225 (e.g., the element 230) is determined based on the usage data at the corresponding time and on the corresponding day.

For example, in the illustrated workflow 200, the tensor 225 contains eight columns (e.g., eight windows of time per day) and five rows (e.g., covering five days). As discussed above, the tensor 225 may generally have any number of rows (e.g., covering any number of days) and any number of columns (e.g., where the time is delineated to any desired level of granularity). For example, in the illustrated tensor 225 each element 230 includes usage data for a three hour window, but in other embodiments, each window may be shorter (e.g., five minute windows, one hour windows, and the like) or longer (e.g., 4 hour windows, 6 hour windows, and the like). In some embodiments, as discussed above, the number of windows of time included, as well as the number of days included, may be hyperparameters of the system or machine learning model (e.g., determined or defined before training the model).

In the illustrated example, the feature component generates a numerical value for each element 230 of the tensor 225 based on the corresponding usage data. For example, in the case of the usage duration attribute, the feature component may generate a continuous value (e.g., between zero and one) indicating the total amount or percentage of time, during the corresponding window, that the user was using the therapy device (e.g., where a value of “1” indicates that the user used the device during the entire window, a value of “0.9” indicates that the user used the device for 90% of the window, and so on). In some aspects, in addition to or instead of a normalized usage value (e.g., between zero and one), the feature component may define the value of each element 230 as the total usage during the corresponding window (e.g., the total number of minutes).

In the illustrated workflow 200, the tensor 225 is a two-dimensional tensor corresponding to one attribute of usage data (e.g., usage duration). In some aspects, as discussed above, the feature component may generate additional channels (e.g., in a three-dimensional tensor) for additional attributes of the usage data. For example, as depicted by the arrow 242, the feature component may perform similar operations to generate the tensor 245 having multiple channels 250. Specifically, as discussed above, each channel 250 in the tensor 245 may correspond to (e.g., contain data for) a respective attribute. In some aspects, each channel 250 may have the same spatial dimensionality. That is, though there may be any number of channels 250 (for any number of attributes), each channel may include the same number of rows (e.g., corresponding to the same number of days) and the same number of columns (e.g., where the data is delineated into the same number of windows of time per day).

For example, one channel may correspond to the usage duration attribute, a second channel may correspond to the residual AHI attribute, a third channel may correspond to the leak measure attribute (e.g., volume of leak over time), a fourth channel may correspond to the pressure attribute (e.g., pressure of the therapy device over time), and the like. In some aspects, the feature component may normalize some or all of the usage data within each window of time (e.g., each column) to a defined scale, such as to continuous values between zero and one. For example, the feature component may clip and/or scale attribute values such as the user's tidal volume or minute ventilation to the same range as other attributes such as usage duration. This normalization may prevent these attributes from dominating the model (due to relatively higher values), allowing the machine learning model itself to learn the relative importance of each attribute.

The workflow 200 may generally be performed during training of the models (e.g., to prepare training exemplars based on prior usage data) as well as during inferencing using the models (e.g., to prepare the input tensors based on usage data during runtime).

### Example Usage Prediction Timeframes

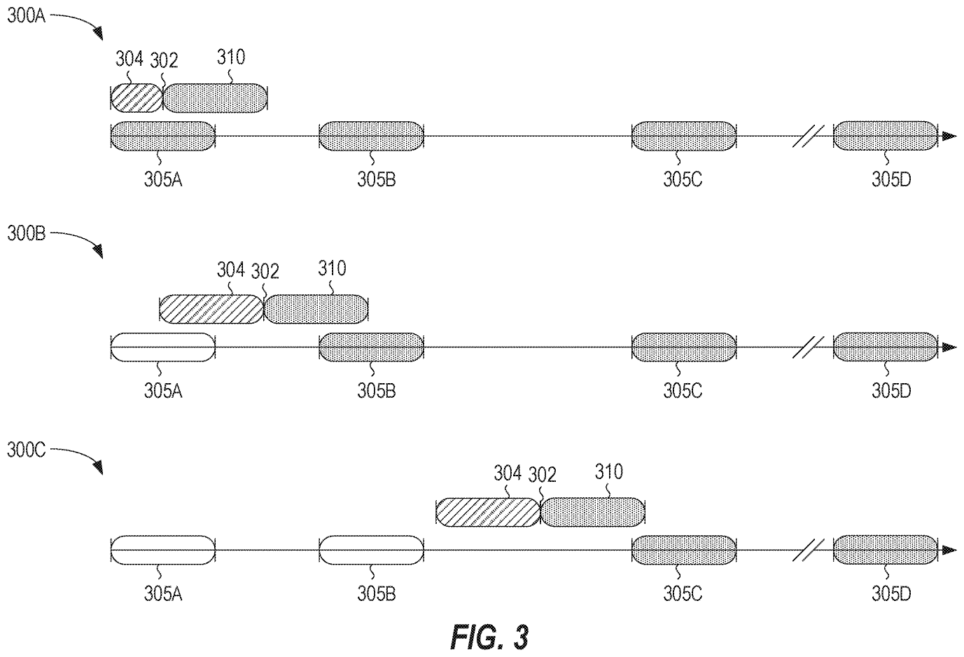

FIG. 3 depicts example timelines 300A-C (collectively, timelines 300) of usage predictions 305A-D and/or 310, according to some embodiments of the present disclosure. In some embodiments, the usage predictions may correspond to the usage predictions 115 of FIG. 1 generated by a machine learning system, such as the machine learning system 110 of FIG. 1.

In the illustrated example, the machine learning system generates up to five usage predictions for a given user at a given time: four fixed or static predictions 305 for defined or fixed windows of time (relative to the user's initial start of therapy) and one dynamic or rolling prediction 310 (corresponding to the current date, as of when the predictions are generated). As discussed above, each prediction may generally relate to the user's usage or engagement in the therapy during a respective future window, such as indicating the probability that the user will still be using the therapy equipment at least a threshold amount during the future window.

For example, each timeline 300 may begin when the user begins respiratory therapy (e.g., when they are first prescribed therapy, when they first receive and/or set up their therapy equipment, and/or when they first engage in therapy by using the therapy equipment). In the illustrated example, the first static prediction 305A may correspond to a first window of time, such as the first thirty days of therapy (e.g., days 0 through 30), the static prediction 305B may correspond to a second window (e.g., from day 60 to day 90), the static prediction 305C may correspond to a third window (e.g., from day 150 to day 180), and the static prediction 305D may correspond to a fourth window (e.g., from day 330 to day 360). Further, on any given Nth day, the rolling prediction 310 may correspond to days N through N+30 (or some other duration). In some embodiments, the predictions for each window of time may be generated using a shared machine learning model, or may be generated using multiple machine learning models (e.g., one model for each window).

More specifically, as illustrated by the timeline 300A, the current date on the timeline 300A is indicated by the line 302 (e.g., midway through the first 30 days). For example, the timeline 300A may be generated on therapy day 14 for a user (e.g., two weeks after the user began therapy). In the illustrated example, the machine learning system may process usage data from a prior window (e.g., indicated by the window 304) for the user to generate predictions on day 14. Specifically, at this time, the user may have five predictions on this date: a prediction 305A regarding the user's compliance or usage during the first window of interest (e.g., days 0 to 30), a prediction 310 regarding the user's usage during days 14 to 44, a prediction 305B regarding the user's usage during the second window of interest (e.g., days 60 to 90), a prediction 305C regarding the user's usage during the third window (e.g., days 150 to 180), and a final prediction 305D regarding the user's usage during the final window of interest (e.g., days 330 to 360).

In the illustrated timeline 300A, the window 304 spans from the first day of therapy to the current day (e.g., day 14), as no information prior to the start of therapy is available. In some embodiments, if the lookback window used by the machine learning system is longer than the current available data, the machine learning system may replace the missing data (for the days prior to therapy start) using predefined values, such as all zeros, all ones, or some other value.

As illustrated by the timeline 300B, the current date for the timeline 300B is also indicated by the line 302 (e.g., between the first and second prediction windows). For example, the timeline 300B may be generated on therapy day 45 for a user (e.g., 45 days after the user began therapy). In the illustrated example, the machine learning system may process usage data from a prior window (e.g., indicated by the window 304) for the user to generate predictions on day 45. Specifically, at this time, the user may have four predictions on this date: a prediction 310 regarding the user's usage during days 45 to 75, a prediction 305B regarding the user's usage during the second window of interest (e.g., days 60 to 90), a prediction 305C regarding the user's usage during the third window (e.g., days 150 to 180), and a final prediction 305D regarding the user's usage during the final window of interest (e.g., days 330 to 360). Notably, as the first window (from days 0 to 30) has already passed, the machine learning system may refrain from generating any prediction for this window. In some embodiments, dynamically refraining from generating this prediction 305A may reduce the computational expense of the machine learning system. In the illustrated example, the window 304 of usage data used to generate the predictions corresponds to the thirty days leading up to the current day (e.g., the usage data from days 15 to 45). However, the particular window used may vary depending on the particular implementation.

Further, as illustrated by the timeline 300C, the current date for the timeline 300C is also indicated by the line 302 (e.g., between the second and third prediction windows). For example, the timeline 300C may be generated on therapy day 100 for a user (e.g., 100 days after the user began therapy). In the illustrated example, the machine learning system may process usage data from a prior window (e.g., indicated by the window 304) for the user to generate predictions on day 100. Specifically, at this time, the user may have three predictions on this date: a prediction 310 regarding the user's usage during days 100 to 130, a prediction 305C regarding the user's usage during the third window (e.g., days 150 to 180), and a final prediction 305D regarding the user's usage during the final window of interest (e.g., days 330 to 360). Notably, as the first and second windows (from days 0 to 30 and from days 60 to 90) have already passed, the machine learning system may refrain from generating any predictions for this windows. In some embodiments, dynamically refraining from generating these prediction 305A and 305B may reduce the computational expense of the machine learning system. In the illustrated example, the window 304 of usage data used to generate the predictions corresponds to the thirty days leading up to the current day (e.g., the usage data from days 70 to 100). However, the particular window used may vary depending on the particular implementation.

In some embodiments, as discussed above, the machine learning system may generate predictions at a variety of points in time. For example, in some embodiments, the machine learning system may generate a new set of predictions for the user periodically (e.g., daily), refraining from generating predictions for any windows (or points) in time that have already passed.

### Example Method for Training Machine Learning Models to Predict Therapy Usage

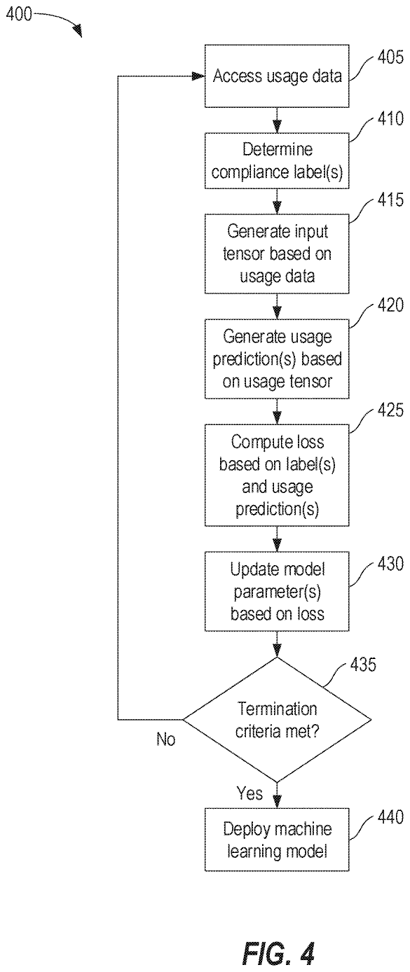

FIG. 4 is a flow diagram depicting an example method 400 for training machine learning models to predict therapy usage, according to some embodiments of the present disclosure. In some embodiments, the method 400 is performed by a machine learning system, such as the machine learning system 110 of FIG. 1.

At block 405, the machine learning system accesses usage data (e.g., the usage data 105 of FIG. 1) for a user of a respiratory therapy system. In some aspects, as discussed above, the usage data corresponds to one or more previous days (e.g., a prior window of time) during which a user was engaged in therapy, such as the past thirty days. The usage data may generally include information relating to a wide variety of respiratory therapy attributes. In some embodiments, as discussed above, some or all of the usage data may include relatively granular data (e.g., including data on a minute-by-minute basis).

At block 410, the machine learning system determines compliance label(s) for the usage data. For example, as discussed above, the machine learning system may evaluate the usage data or other information to determine whether the user was compliant with their respiratory therapy during one or more defined windows of time (e.g., the static and/or rolling windows discussed above with reference to FIG. 3). Although the illustrated example depicts determining usage compliance, in some embodiments, the machine learning system may generally determine any usage metric that the model is being trained to predict.

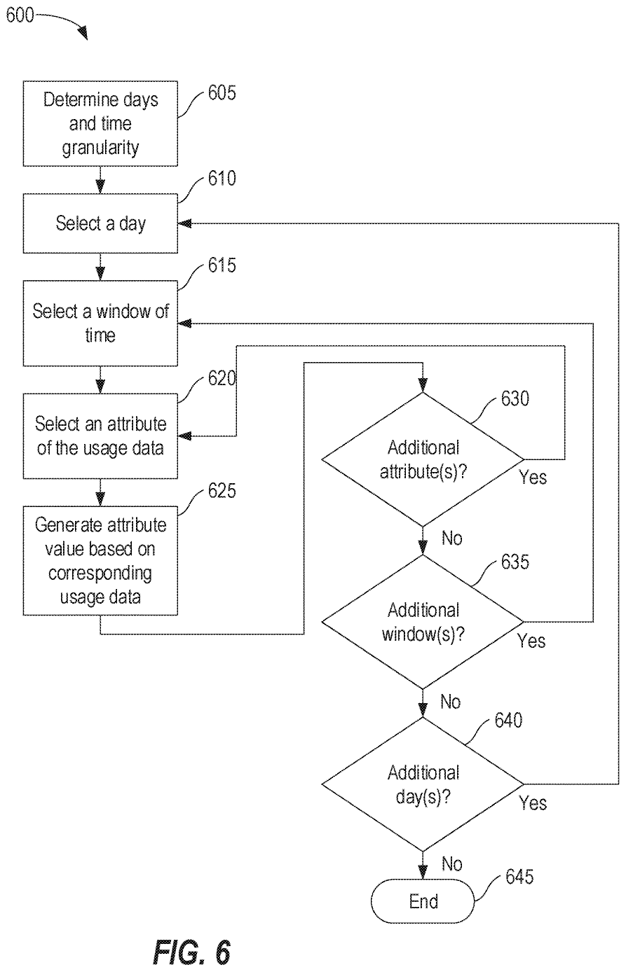

At block 415, the machine learning system generates an input tensor (also referred to as a usage tensor, a feature tensor, and/or an attribute tensor in some aspects, as discussed above) based on some or all of the usage data. For example, as discussed above, the machine learning system may evaluate the usage data for a window of time culminating at the current date in order to generate a tensor (e.g., the tensor 225 and/or 245 of FIG. 2). In some aspects, as discussed above, the spatial dimensions of the tensor may correspond to the number of days in the window and the number of time buckets per day (e.g., where each element indicates the relevant usage data during a given time on a given day). In some embodiments, as discussed above, the tensor may have a depth greater than one (e.g., multiple channels for multiple attributes). One example method of generating the input tensor is discussed below in more detail with reference to FIG. 6.

At block 420, the machine learning system generates one or more usage predictions (e.g., the usage predictions 115 of FIG. 1) based on the usage tensor. For example, as discussed above, the machine learning system may process the tensor using one or more machine learning models (e.g., CNNs). As discussed above, the usage predictions may generally indicate the predicted usage of the respiratory therapy system, by the user, at one or more points (or during one or more windows) at a future point in time, relative to the time corresponding to the input tensor. For example, the predictions may indicate the probability that the user will be deemed “compliant” during the future windows based on one or more usage-based compliance criteria.

At block 425, the machine learning system computes one or more losses based on the determined compliance label(s) (or other label(s)) and the generated usage prediction(s) (e.g., based on the difference between the prediction(s) and the label(s)). Generally, a variety of loss formulations may be used, depending on the particular implementation. For example, in some aspects, the machine learning system may use a cross-entropy loss, such that predictions that are significantly different from the ground truth labels result in a relatively high loss, as compared to predictions that are more similar to the ground truth labels.

At block 430, the machine learning system updates the parameter(s) of the machine learning model based on the loss(es). Generally, the particular operations used to update the parameters may vary depending on the particular implementation. For example, in some cases (such as if the model comprises a CNN), the machine learning system may use backpropagation to iteratively refine the model parameters based on the loss(es).

At block 435, the machine learning system determines whether one or more training termination criteria are met. The particular termination criteria may vary depending on the particular implementation. For example, in some embodiments, the machine learning system may determine whether a one or more targets have been reached (e.g., a defined number of training iterations completed, a defined model accuracy being met, and the like.

If, at block 435, the machine learning system determines that the termination criteria are not met, the method 400 returns to block 405 to access another set of usage data (e.g., for a different user, or for the same user at a different time) to continue training the model(s). If, at block 435, the machine learning system determines that the termination criteria are met, the method 400 continues to block 440. Although the illustrated example depicts a sequential process (e.g., training the model on individual exemplars, such as using stochastic gradient descent) for conceptual clarity, in some aspects, the machine learning system may additionally or alternatively train the model on multiple exemplars in parallel (e.g., using batch gradient descent).

At block 440, the machine learning system deploys the machine learning model for runtime user. Generally, deploying the model may include performing any operations to prepare or provide the model for inferencing. For example, the machine learning system may instantiate the model locally for local use, or may transmit the model parameters to a second system for runtime use.

### Example Method for Using Machine Learning Models to Predict Therapy Usage

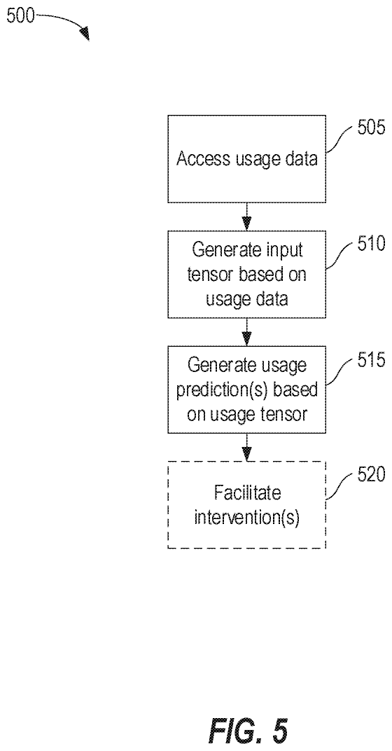

FIG. 5 is a flow diagram depicting an example method 500 for using machine learning models to predict therapy usage, according to some embodiments of the present disclosure. In some embodiments, the method 500 is performed by a machine learning system, such as the machine learning system 110 of FIG. 1.

At block 505, the machine learning system accesses usage data (e.g., the usage data 105 of FIG. 1) for a user of a respiratory therapy system. In some aspects, as discussed above, the usage data corresponds to one or more previous days (e.g., a prior window of time) during which a user was engaged in therapy, such as the past thirty days. The usage data may generally include information relating to a wide variety of respiratory therapy attributes. In some embodiments, as discussed above, some or all of the usage data may include relatively granular data (e.g., including data on a minute-by-minute basis).

At block 510, the machine learning system generates an input tensor (also referred to as a usage tensor, a feature tensor, and/or an attribute tensor in some aspects, as discussed above) based on some or all of the usage data. For example, as discussed above, the machine learning system may evaluate the usage data for a window of time culminating at the current date in order to generate a tensor (e.g., the tensor 225 and/or 245 of FIG. 2). In some aspects, as discussed above, the spatial dimensions of the tensor may correspond to the number of days in the window and the number of time buckets per day (e.g., where each element indicates the relevant usage data during a given time on a given day). In some embodiments, as discussed above, the tensor may have a depth greater than one (e.g., multiple channels for multiple attributes). One example method of generating the input tensor is discussed below in more detail with reference to FIG. 6.

At block 515, the machine learning system generates one or more usage predictions (e.g., the usage predictions 115 of FIG. 1) based on the usage tensor. For example, as discussed above, the machine learning system may process the tensor using one or more machine learning models (e.g., CNNs). As discussed above, the usage predictions may generally indicate the predicted usage of the respiratory therapy system, by the user, at one or more points (or during one or more windows) at a future point in time, relative to the time corresponding to the input tensor. For example, the predictions may indicate the probability that the user will be deemed “compliant” during the future windows based on one or more usage-based compliance criteria.

At block 520, the machine learning system optionally facilitates one or more interventions (e.g., the interventions 120 of FIG. 1) for the user based on the usage prediction(s). For example, as discussed above, the machine learning system may determine whether the usage predictions satisfy one or more criteria (e.g., whether the user is sufficiently likely to use the respiratory therapy system at least a threshold amount during one or more future windows of time), and may initiate intervention(s) if the criteria are not satisfied (e.g., the user is predicted to stop using the therapy system). As another example, the machine learning system may initiate interventions if the criteria are satisfied (e.g., ordering or suggesting resupply materials if the prediction is sufficiently high, indicating a good probability that the user will remain engaged in therapy and therefore will be due for such resupply in the future). As discussed above, the particular interventions used may vary depending on the particular implementation.

### Example Method for Generating Granular Input Tensors

FIG. 6 is a flow diagram depicting an example method for generating granular input tensors, according to some embodiments of the present disclosure. In some embodiments, the method 600 is performed by a machine learning system, such as the machine learning system 110 of FIG. 1. In some embodiments, the method 600 provides additional detail for the block 415 of FIG. 4 and/or the block 510 of FIG. 5.

At block 605, the machine learning system determines the number of days and time granularity to use to define feature tensors. For example, as discussed above, the machine learning system may determine the number of days that should be included in the input data (e.g., the number of rows in the tensor) and the granularity of the windows of time (e.g., the number of columns in the tensor). In some embodiments, as discussed above, the number of days and windows of time may be hyperparameters (e.g., specified by a data scientist) for training and/or inferencing using the machine learning model.

At block 610, the machine learning system selects a day reflected in the usage data and included in the determined window of days (e.g., included in the N days leading up to a current or index day, as discussed above). Generally, the machine learning system may use a variety of operations to select the day, including randomly or pseudo-randomly, as the machine learning system may select and process data from each day in the prediction window during the method 600.

At block 615, the machine learning system selects a window of time for evaluation. For example, if the time granularity is one hour (e.g., there are twenty-four columns in the tensor and the usage data is to be aggregated and evaluated on a per-hour basis), the machine learning system may select a one hour window (e.g., from midnight to one in the morning, from two to three in the afternoon, and the like). Generally, the machine learning system may use a variety of operations to select the window of time, including randomly or pseudo-randomly, as the machine learning system may select and process data from each window of time in each day during the method 600.

At block 620, the machine learning system selects an attribute of the usage data. For example, as discussed above, the usage data may include information for a variety of attributes, and the machine learning system may use any number of these attributes as separate input features for the model. Generally, the machine learning system may use a variety of operations to select the attribute, including randomly or pseudo-randomly, as the machine learning system may select and process data for each attribute during the method 600.

At block 625, the machine learning system generates an attribute value for the selected attribute based on corresponding usage data (e.g., usage data corresponding to the selected window of time on the selected day). That is, the machine learning system may generate an attribute value (e.g., a numeric value) representing the selected attribute during the selected window. In some aspects, as discussed above, the machine learning system may normalize the attribute value to a defined range (e.g., between zero and one). In this way, the machine learning system can generate the value, in the input tensor, for the element that corresponds to the selected attribute (e.g., in the corresponding channel) at the selected time and day (e.g., at the corresponding spatial indices in the tensor).

At block 630, the machine learning system determines whether there is at least one additional attribute that has not yet been evaluated to generate a corresponding channel in the tensor. If so, the method 600 returns to block 620 to select another attribute for analysis. If, at block 630, the machine learning system determines that no additional attributes remain, the method 600 continues to block 635. Although the illustrated example depicts a sequential process (e.g., selecting and evaluating each attribute iteratively) for conceptual clarity, in some aspects, the machine learning system may additionally or alternatively evaluate some or all of the attributes entirely or partially in parallel.

At block 635, the machine learning system determines whether there is at least one additional window of time that has not yet been evaluated with respect to the selected day to generate a corresponding set of attribute values (across one or more channels). If so, the method 600 returns to block 615 to select another window of time for analysis. If, at block 635, the machine learning system determines that no additional windows of time remain for the selected day, the method 600 continues to block 640. Although the illustrated example depicts a sequential process (e.g., selecting and evaluating each window of time iteratively, and then evaluating each attribute with respect to the selected window of time) for conceptual clarity, in some aspects, the machine learning system may additionally or alternatively evaluate some or all of the windows of time entirely or partially in parallel.

At block 640, the machine learning system determines whether there is at least one additional day that has not yet been evaluated to generate a corresponding row in the input tensor. If so, the method 600 returns to block 610 to select another day for analysis. If, at block 640, the machine learning system determines that no additional days remain in the lookback window, the method 600 terminates at block 645. Although the illustrated example depicts a sequential process (e.g., selecting and evaluating each day iteratively, and then evaluating each attribute with respect to the each window of time in the day) for conceptual clarity, in some aspects, the machine learning system may additionally or alternatively evaluate some or all of the days entirely or partially in parallel.

Although the illustrated example depicts generating an input tensor corresponding to one user for conceptual clarity, in some embodiments, the method 600 may be performed any number of times for any number of users (in sequence, or entirely or partially in parallel). In some aspects, the input tensors for multiple users may be combined or aggregated (e.g., concatenated) to form a batch of data to be used as input to the model (e.g., in a batch processing implementation).

As discussed above, dynamically generating the multidimensional tensor to represent the usage data can enable use of substantially more accurate and efficient machine learning models, as compared to some conventional approaches.

### Example Method for Predicting Usage Using Machine Learning

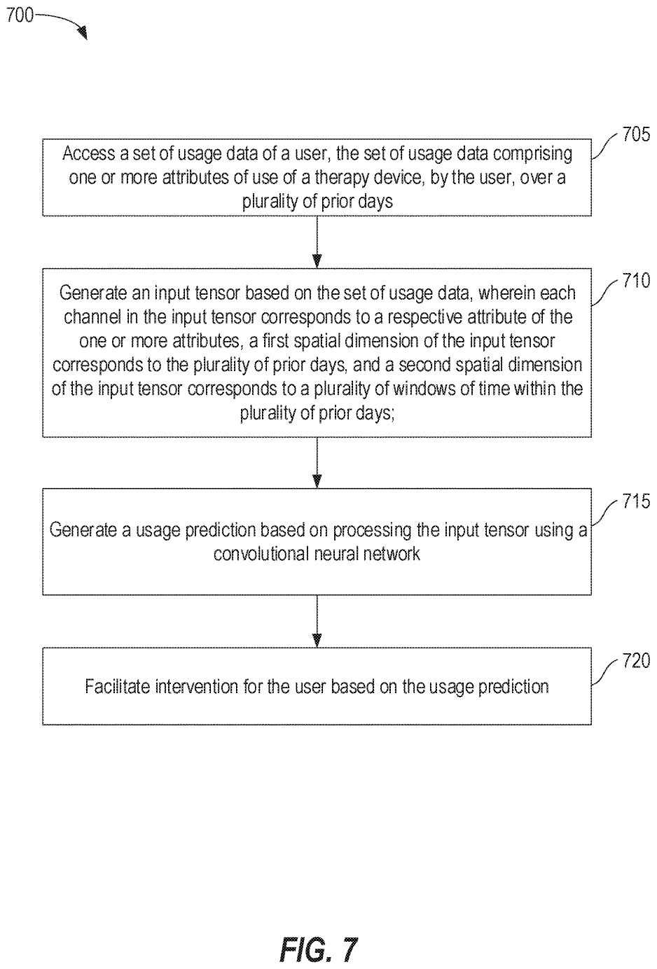

FIG. 7 is a flow diagram depicting an example method 700 for predicting usage using machine learning, according to some embodiments of the present disclosure. In some embodiments, the method 700 is performed by a machine learning system, such as the machine learning system 110 of FIG. 1.

At block 705, a set of usage data (e.g., the usage data 105 of FIG. 1) of a user is accessed, the set of usage data comprising one or more attributes of use of a therapy device, by the user, over a plurality of prior days.

At block 710, an input tensor (e.g., the tensor 225 and/or 245 of FIG. 2) is generated based on the set of usage data, wherein each channel (e.g., the channels 250 of FIG. 2) in the input tensor corresponds to a respective attribute of the one or more attributes, a first spatial dimension of the input tensor (e.g., the rows 240 of FIG. 2) corresponds to the plurality of prior days, and a second spatial dimension of the input tensor (e.g., the columns 235 of FIG. 2) corresponds to a plurality of windows of time within the plurality of prior days.

At block 715, a usage prediction (e.g., the usage prediction 115 of FIG. 1) is generated based on processing the input tensor using a convolutional neural network.

At block 720, intervention (e.g., the intervention(s) 120 of FIG. 1) for the user is facilitated based on the usage prediction.

### Example Processing System for Usage Prediction Machine Learning

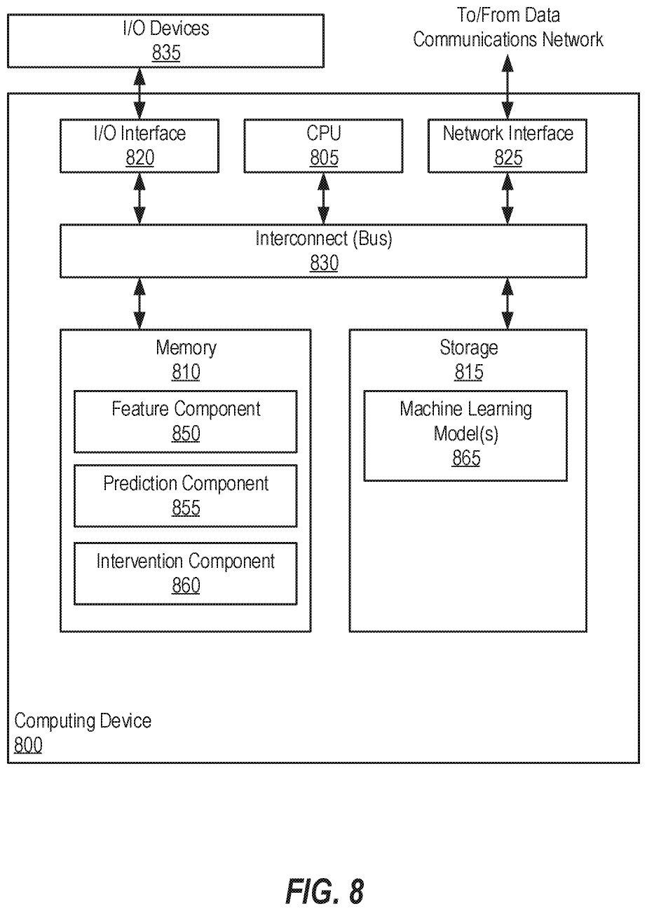

FIG. 8 depicts an example computing device 800 configured to perform various aspects of the present disclosure, according to some embodiments disclosed herein. Although depicted as a physical device, in embodiments, the computing device 800 may be implemented using virtual device(s), and/or across a number of devices (e.g., in a cloud environment). In one embodiment, the computing device 800 corresponds to any element or aspect of machine learning system, such as the machine learning system 110 of FIG. 1 and/or the machine learning system discussed above with reference to FIGS. 2-7.

As illustrated, the computing device 800 includes a CPU 805, memory 810, storage 815, a network interface 825, and one or more input/output (I/O) interfaces 820. In the illustrated embodiment, the CPU 805 retrieves and executes programming instructions stored in memory 810, as well as stores and retrieves application data residing in storage 815. The CPU 805 is generally representative of a single CPU and/or GPU, multiple CPUs and/or GPUs, a single CPU and/or GPU having multiple processing cores, and the like. The memory 810 is generally included to be representative of a random access memory. Storage 815 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 835 (such as keyboards, monitors, etc.) are connected via the I/O interface(s) 820. Further, via the network interface 825, the computing device 800 can be communicatively coupled with one or more other devices and components (e.g., via a network, which may include the Internet, local network(s), and the like). As illustrated, the CPU 805, memory 810, storage 815, network interface(s) 825, and I/O interface(s) 820 are communicatively coupled by one or more buses 830.

In the illustrated embodiment, the memory 810 includes a feature component 850, a prediction component 855, and an intervention component 860, which may perform one or more embodiments discussed above. Although depicted as discrete components for conceptual clarity, in embodiments, the operations of the depicted components (and others not illustrated) may be combined or distributed across any number of components. Further, although depicted as software residing in memory 810, in embodiments, the operations of the depicted components (and others not illustrated) may be implemented using hardware, software, or a combination of hardware and software. Additionally, in some embodiments, additional components may be present, such as training components for training or refining the machine learning model(s).

In some embodiments, the feature component 850 (which may correspond to the feature component 125 of FIG. 1) can be used to generate usage tensors (e.g., the tensors 225 and/or 245 of FIG. 2) based on input usage data, as discussed above. For example, the feature component 850 may generate, for each defined window of time on each day in a lookback period, generate feature or attribute values (e.g., across one or more channels of the tensor) based on corresponding usage data for the window and day.

In some embodiments, the prediction component 855 (which may correspond to the prediction component 130 of FIG. 1) can be used to generate usage predictions (e.g., the usage predictions 115 of FIG. 1), as discussed above. For example, the prediction component 855 may process the usage tensors (generated by the feature component 850) using one or more machine learning models (e.g., the machine learning models 865), such as convolutional neural networks, to generate predictions relating to the future usage of a respiratory therapy system by a patient.

In some embodiments, the intervention component 860 (which may correspond to the intervention component 135 of FIG. 1) can be used to selectively and/or dynamically generate interventions based on predicted usage, as discussed above. For example, the intervention component 860 may compare the predicted usage against one or more criteria (e.g., thresholds), and may take various actions such as transmitting coaching materials, alerting a healthcare provider, requesting resupply materials, and the like.

In the illustrated example, the storage 815 includes one or more machine learning models 865, which may generally correspond to models (e.g., CNNs) trained to generate usage predictions based on input tensors, as discussed above. Although depicted as residing in storage 815, the depicted data may be stored in any suitable location, including memory 810.

Generally, the depicted components (and others not depicted) in memory 810 may be used to implement one or more embodiments discussed above.

### Example Clauses

Clause 1: A method, comprising: accessing a set of usage data of a user, the set of usage data comprising one or more attributes of use of a therapy device, by the user, over a plurality of prior days; generating an input tensor based on the set of usage data, wherein: each channel in the input tensor corresponds to a respective attribute of the one or more attributes, a first spatial dimension of the input tensor corresponds to the plurality of prior days, and a second spatial dimension of the input tensor corresponds to a plurality of windows of time within the plurality of prior days; generating a usage prediction based on processing the input tensor using a convolutional neural network; and facilitating intervention for the user based on the usage prediction.
Clause 2: A method according to Clause 1, wherein: the therapy device comprises a respiratory therapy device for a respiratory therapy engaged in by the user, and the set of usage data indicates at least one of: (i) how long the user used the respiratory therapy device during one or more of the plurality of prior days, (ii) a residual apnea-hypopnea index (AHI) of the user during one or more of the plurality of prior days, (iii) a leak measure of the respiratory therapy during one or more of the plurality of prior days, or (iv) a pressure of the respiratory therapy device during one or more of the plurality of prior days.
Clause 3: A method according to any of Clauses 1-2, wherein generating the input tensor comprises, for each respective window of time of the plurality of windows of time corresponding to a first day of the plurality of prior days: determining a respective attribute value for a first attribute, of the one or more attributes, with respect to the user and the therapy device during the respective window of time on the first day; and updating a value of a respective element of the input tensor based on the respective attribute value, wherein the corresponding element corresponds to the first attribute during the respective window of time on the first day.
Clause 4: A method according to any of Clauses 1-3, wherein generating the input tensor comprises, for each respective element of the input tensor, normalizing a corresponding value, determined from the set of usage data, to a value between zero and one, inclusively.
Clause 5: A method according to any of Clauses 1-4, wherein the input tensor comprises, for each respective day of the plurality of prior days, a respective row.
Clause 6: A method according to any of Clauses 1-3, wherein the input tensor comprises, for each respective window of time, a respective column.
Clause 7: A method according to Clause 6, wherein each respective column corresponds to a respective hour.
Clause 8: A method according to any of Clauses 1-7, wherein a number of the plurality of prior days and a number of the plurality of windows of time are hyperparameters of the convolutional neural network.
Clause 9: A method according to any of Clauses 1-8, wherein the usage prediction comprises a prediction as to whether usage of the therapy device, by the user, will satisfy one or more defined criteria with respect to a plurality of future days.
Clause 10: A system, comprising: a memory comprising computer-executable instructions; and one or more processors configured to execute the computer-executable instructions and cause the processing system to perform a method in accordance with any one of Clauses 1-9.
Clause 11: A system, comprising means for performing a method in accordance with any one of Clauses 1-9.
Clause 12: A non-transitory computer-readable medium comprising computer-executable instructions that, when executed by one or more processors of a processing system, cause the processing system to perform a method in accordance with any one of Clauses 1-9.
Clause 13: A computer program product embodied on a computer-readable storage medium comprising code for performing a method in accordance with any one of Clauses 1-9.

### Additional Considerations

One or more elements or aspects or steps, or any portion(s) thereof, from one or more of any of claims below can be combined with one or more elements or aspects or steps, or any portion(s) thereof, from one or more of any of the other claims below or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g., an amount of storage space consumed by a user or a number of virtualized systems instantiated by the user). A user can access any of the resources that reside in the cloud at any time, and from anywhere across the Internet. In context of the present invention, a user may access applications or systems (e.g., the engagement prediction system) or related data available in the cloud. For example, the engagement prediction system could execute on a computing system in the cloud and train and use machine learning models to predict user engagement with respect to media content and/or respiratory therapy, as discussed above. In such a case, the engagement prediction system could receive and process engagement data, and store the models and predictions at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
accessing a set of usage data of a user, the set of usage data comprising one or more attributes of use of a therapy device, by the user, over a plurality of prior days;
generating an input tensor based on the set of usage data, wherein:
each channel in the input tensor corresponds to a respective attribute of the one or more attributes,
a first spatial dimension of the input tensor corresponds to the plurality of prior days, and
a second spatial dimension of the input tensor corresponds to a plurality of windows of time within the plurality of prior days;
generating a usage prediction based on processing the input tensor using a convolutional neural network; and
facilitating intervention for the user based on the usage prediction.

2. The method of claim 1, wherein:
the therapy device comprises a respiratory therapy device for a respiratory therapy engaged in by the user, and
the set of usage data indicates at least one of:
(i) how long the user used the respiratory therapy device during one or more of the plurality of prior days,
(ii) a residual apnea-hypopnea index (AHI) of the user during one or more of the plurality of prior days,
(iii) a leak measure of the respiratory therapy during one or more of the plurality of prior days, or
(iv) a pressure of the respiratory therapy device during one or more of the plurality of prior days.

3. The method of claim 1, wherein generating the input tensor comprises, for each respective window of time of the plurality of windows of time corresponding to a first day of the plurality of prior days:
determining a respective attribute value for a first attribute, of the one or more attributes, with respect to the user and the therapy device during the respective window of time on the first day; and
updating a value of a respective element of the input tensor based on the respective attribute value, wherein the corresponding element corresponds to the first attribute during the respective window of time on the first day.

4. The method of claim 1, wherein generating the input tensor comprises, for each respective element of the input tensor, normalizing a corresponding value, determined from the set of usage data, to a value between zero and one, inclusively.

5. The method of claim 1, wherein the input tensor comprises, for each respective day of the plurality of prior days, a respective row.

6. The method of claim 1, wherein the input tensor comprises, for each respective window of time, a respective column.

7. The method of claim 6, wherein each respective column corresponds to a respective hour.

8. The method of claim 1, wherein a number of the plurality of prior days and a number of the plurality of windows of time are hyperparameters of the convolutional neural network.

9. The method of claim 1, wherein the usage prediction comprises a prediction as to whether usage of the therapy device, by the user, will satisfy one or more defined criteria with respect to a plurality of future days.

10. One or more non-transitory computer-readable media collectively or individually comprising computer-executable instructions that, when executed by one or more processors of one or more processing systems, cause the one or more processing systems to collectively or individually perform an operation comprising:
accessing a set of usage data of a user, the set of usage data comprising one or more attributes of use of a therapy device, by the user, over a plurality of prior days;
generating an input tensor based on the set of usage data, wherein:
each channel in the input tensor corresponds to a respective attribute of the one or more attributes,
a first spatial dimension of the input tensor corresponds to the plurality of prior days, and
a second spatial dimension of the input tensor corresponds to a plurality of windows of time within the plurality of prior days;
generating a usage prediction based on processing the input tensor using a convolutional neural network; and
facilitating intervention for the user based on the usage prediction.

11. The one or more non-transitory computer-readable media of claim 10, wherein:
the therapy device comprises a respiratory therapy device for a respiratory therapy engaged in by the user, and
the set of usage data indicates at least one of:
(i) how long the user used the respiratory therapy device during one or more of the plurality of prior days,
(ii) a residual apnea-hypopnea index (AHI) of the user during one or more of the plurality of prior days,
(iii) a leak measure of the respiratory therapy during one or more of the plurality of prior days, or
(iv) a pressure of the respiratory therapy device during one or more of the plurality of prior days.

12. The one or more non-transitory computer-readable media of claim 10, wherein generating the input tensor comprises, for each respective window of time of the plurality of windows of time corresponding to a first day of the plurality of prior days:
determining a respective attribute value for a first attribute, of the one or more attributes, with respect to the user and the therapy device during the respective window of time on the first day; and
updating a value of a respective element of the input tensor based on the respective attribute value, wherein the corresponding element corresponds to the first attribute during the respective window of time on the first day.

13. The one or more non-transitory computer-readable media of claim 10, wherein generating the input tensor comprises, for each respective element of the input tensor, normalizing a corresponding value, determined from the set of usage data, to a value between zero and one, inclusively.

14. The one or more non-transitory computer-readable media of claim 10, wherein:
the input tensor comprises, for each respective day of the plurality of prior days, a respective row,
the input tensor comprises, for each respective window of time, a respective column, and
a number of the plurality of prior days and a number of the plurality of windows of time are hyperparameters of the convolutional neural network.

15. The one or more non-transitory computer-readable media of claim 10, wherein the usage prediction comprises a prediction as to whether usage of the therapy device, by the user, will satisfy one or more defined criteria with respect to a plurality of future days.

16. A system, comprising:
one or more memories collectively or individually comprising computer-executable instructions; and
one or more processors configured to, individually or collectively, execute the computer-executable instructions and cause the system to perform an operation comprising:
accessing a set of usage data of a user, the set of usage data comprising one or more attributes of use of a therapy device, by the user, over a plurality of prior days;
generating an input tensor based on the set of usage data, wherein:
each channel in the input tensor corresponds to a respective attribute of the one or more attributes,
a first spatial dimension of the input tensor corresponds to the plurality of prior days, and
a second spatial dimension of the input tensor corresponds to a plurality of windows of time within the plurality of prior days;
generating a usage prediction based on processing the input tensor using a convolutional neural network; and
facilitating intervention for the user based on the usage prediction.

17. The system of claim 16, wherein:
the therapy device comprises a respiratory therapy device for a respiratory therapy engaged in by the user, and
the set of usage data indicates at least one of:
(i) how long the user used the respiratory therapy device during one or more of the plurality of prior days,
(ii) a residual apnea-hypopnea index (AHI) of the user during one or more of the plurality of prior days,
(iii) a leak measure of the respiratory therapy during one or more of the plurality of prior days, or
(iv) a pressure of the respiratory therapy device during one or more of the plurality of prior days.

18. The system of claim 16, wherein generating the input tensor comprises, for each respective window of time of the plurality of windows of time corresponding to a first day of the plurality of prior days:
determining a respective attribute value for a first attribute, of the one or more attributes, with respect to the user and the therapy device during the respective window of time on the first day; and
updating a value of a respective element of the input tensor based on the respective attribute value, wherein the corresponding element corresponds to the first attribute during the respective window of time on the first day.

19. The system of claim 16, wherein generating the input tensor comprises, for each respective element of the input tensor, normalizing a corresponding value, determined from the set of usage data, to a value between zero and one, inclusively.

20. The system of claim 16, wherein:
the input tensor comprises, for each respective day of the plurality of prior days, a respective row,
the input tensor comprises, for each respective window of time, a respective column, and
a number of the plurality of prior days and a number of the plurality of windows of time are hyperparameters of the convolutional neural network.
